SEQUENCE LISTING (8 sequences)

SEQ ID NO: 1 - protein, 22 aa, synthetic construct
MDMRVPAQLLGLLLLWLRGARC

SEQ ID NO: 2 - protein, 19 aa, synthetic construct
MAWALLLLTLLTQGTGSWA

SEQ ID NO: 3 - protein, 19 aa, synthetic construct
MTCSPLLLTLLIHCTGSWA

SEQ ID NO: 4 - protein, 20 aa, synthetic construct
MEAPAQLLFLLLLWLPDTTG

SEQ ID NO: 5 - protein, 19 aa, synthetic construct
MEWTWRVLFLVAAATGAHS

SEQ ID NO: 6 - protein, 20 aa, synthetic construct
METPAQLLFLLLLWLPDTTG

SEQ ID NO: 7 - protein, 19 aa, synthetic construct
MKHLWFFLLLVAAPRWVLS

SEQ ID NO: 8 - protein, 19 aa, synthetic construct
MEWSWVFLFFLSVTTGVHS

(19) United States
(12) Patent Application Publication
(10) Pub. No.: US 2025/0188495 A1
(43) Pub. Date: Jun. 12, 2025

(54) OPTIMIZED TRANSFECTION PROTOCOL

(71) Applicant: AMGEN INC., Thousand Oaks, CA (US)

(72) Inventors: Danqing LI, Newbury Park, CA (US); Danyang GONG, Arcadia, CA (US); Fernando GARCES, San Francisco, CA (US)

(73) Assignee: AMGEN INC., Thousand Oaks, CA (US)

(21) Appl. No.: 18/845,273

(22) PCT Filed: Mar. 8, 2023
(86) PCT No.: PCT/US2023/063953

§ 371 Date: Sep. 9, 2024

Related U.S. Application Data
(60) Provisional application No. 63/317,959, filed on Mar. 9, 2022.

(51) Int. Cl. C12N 15/88; C07K 16/00

(52) U.S. Cl. CPC C12N 15/88; C07K 16/00; C07K 2317/14

(57) ABSTRACT

The present invention relates to an optimized method of transfecting cells that requires less steps and less DNA than previously disclosed methods. In addition, the method allows for less days in the laboratory for scientists.

This claims the benefit of U.S. Provisional Patent Application No. 63/317,959, filed Mar. 9, 2022, which is incorporated herein by reference in its entirety.

### INCORPORATION BY REFERENCE

Incorporated by reference in its entirety is a sequence listing in XML format, identifiable by the following file properties: Filename: 10071-WO01-SEC.XML; File size: 7,813 bytes; Created: Feb. 17, 2023.

### FIELD OF THE INVENTION

The present invention relates to an optimized method of transfecting cells that requires less steps and less DNA than previously disclosed methods.

### BACKGROUND TO THE INVENTION

In 2013, Jager et al (Jager V, Bussow K, Wagner A, Weber S, Hust M, Frenzel A. Schirrmann T. High level transient production of recombinant antibodies and antibody fusion proteins in HEK293 cells. BMC Biotechnol 2013; 13:52.) reported an optimized HEK293-6E protocol for the expression of monospecific scFv-Fc antibody. To date, this paper has been cited 163 times in both academic and industrial publications suggesting a widely application of this protocol. Briefly, in this protocol, cells are transfected with 0.5 mg/L DNA using PEI reagent, followed by feed with Sheff-Vax+Tryptone N1 one day later, a second feed with glucose on the 3rd day, and addition of 3.75 mM of sodium valproic (VPA) on the 4th day after transfection. Even though Jager's protocol was shown to support recombinant protein expression in high yield and relatively high throughput, we rationalized that this protocol could be further optimized. Moreover, Jager's protocol only examined scFv-Fc antibodies. In recent years, multispecific antibodies (Multispecifics) which can target multiple targets simultaneously show growing interest in therapeutic development. Therefore, new HEK293-6E protocols are necessary to be optimized and validated for expressing quality Multispecifics with high yield and throughput.

Our goal is to develop HEK293-6E protocol(s) that can support high yield of Multispecifics, save resource and increase throughput.

Transient transfection of Human Embryonic Kidney 293 (HEK293-6E) cells is widely used in the biotechnology industry to quickly produce recombinant protein during pre-clinical development. In 2013, Jager et al reported a HEK293-6E protocol for high-throughput expression of monospecific scFv-Fc antibody. The present invention is an improvement upon the Jager protocol. Compared to Jager's protocol, Grace_v1 requires 50% less DNA and no FBS, reduces experimental step by 1, meanwhile shows comparable protein yield. With a goal of further optimizing HEK293-6E protocol, we designed 9 new protocols with variations in key steps of Grace_v1. After evaluation of these 9 protocols together with Grace_v1 in expressing a large panel of mAbs and Multispecifics, two new protocols (Grace_v2 and v3) show comparable protein yield and quality as Grace_v1. Surprisingly, these two new protocols don't need the addition of VPA after transfection. Omitting VPA not only saves resource but also significantly increases experiment throughput and improves flexibility. Furthermore, compared to Jager's protocol, Grace_v2 and _v3 can save resource (50% less DNA, no FBS) and increase throughput (2 experimental steps less).

### SUMMARY OF THE INVENTION

In one aspect, the present invention is directed to a method for transfecting a population of mammalian cells with DNA encoding a target molecule, the method comprising the following steps:
(a) providing the cells in cell culture media;
(b) performing a transfection step by contacting the cells with a liposome/DNA complex; wherein the mg DNA:cell number ratio is about 0.25 mg:1×10⁹ cells;
(c) at about 40-56 hours after the transfection step, adding the following to the cell culture media in any order:

Tryptone N1 to a final concentration of about 4.5-5.5 g/L,
glucose, to a final concentration of about 4.0-5.0 g/L, and
about 0.8-12 volumes of fresh cell culture media;
(d) at about 144-192 hours after the transfection step, harvesting the target molecule from the cell culture media.

In one embodiment, step (c) is performed at about 48 hours after the transfection step.

In one aspect, the present invention is directed to a method for transfecting a population of mammalian cells with DNA encoding a target molecule, the method comprising the following steps:
(a) providing the cells in cell culture media;
(b) performing a transfection step by contacting the cells with a liposome/DNA complex;
wherein the mg DNA:cell number ratio is about 0.25 mg:1×10⁹ cells; (c) at about 2-6 hours after the transfection step, adding the following to the cell culture media in any order:

Tryptone N1 to a final concentration of about 45-5.5 g/L,
glucose to a final concentration of about 4.0-5.0 g/L, and
about 0.8-1.2 volumes of fresh cell culture media;
(d) at about 144-192 hours after the transfection step, harvesting the target molecule from the cell culture media.

In one embodiment, step (c) is performed at about 4 hours after the transfection step.

In one embodiment, the cells are suspension cells.

In one embodiment, wherein the cells are adherent cells.

In one embodiment, the cells are selected from the group consisting of CHO cells, CHOK1 cells, DXB-11 cells, DG-44 cells, COS-7 cells, HEK293-6E cells, BHK cells, TM4 cells, CV1 cells, VERO-76 cells, HELA cells, MDCK cells, BRL 3A cells, W138 cells, Hep G2 cells, MMT cells, TRI cells, MRC 5 cells, and FS4 cells.

In one embodiment, the cells are HEK293-6E cells.

In one embodiment, the cells are seeded at about 1×10⁵-1×10⁷ cells/ml.

In one embodiment, the cells are seeded at about 1×10⁶/ml.

In one embodiment, the final concentration of Tryptone N1 is about 5.0 g/L.

In one embodiment, the final concentration of glucose is about 4.5 g/L.

In one embodiment, about 1 volume of fresh cell culture media is added.

In one embodiment, at about 88-104 hours after the transfection step, valproic acid is added to a final concentration of about 3.5-4.0 mM.

In one embodiment, the final concentration of valproic acid is about 3.75 mM.

In one embodiment, step (d) is performed at about 168 hours after the transfection step.

In one embodiment, the valproic acid is added at about 96 hours after the transfection step.

In one embodiment, the target molecule is a multispecific antigen binding protein.

### BRIEF DESCRIPTION OF THE DRAWINGS

FIG. 1 depicts a comparison to Jager's protocol. Grace_v1 showed higher antibody expression (A), better cell growth (B) and comparable cell viability (C).

FIG. 2 depicts a Schematic of molecules (A) and experimental design (B).

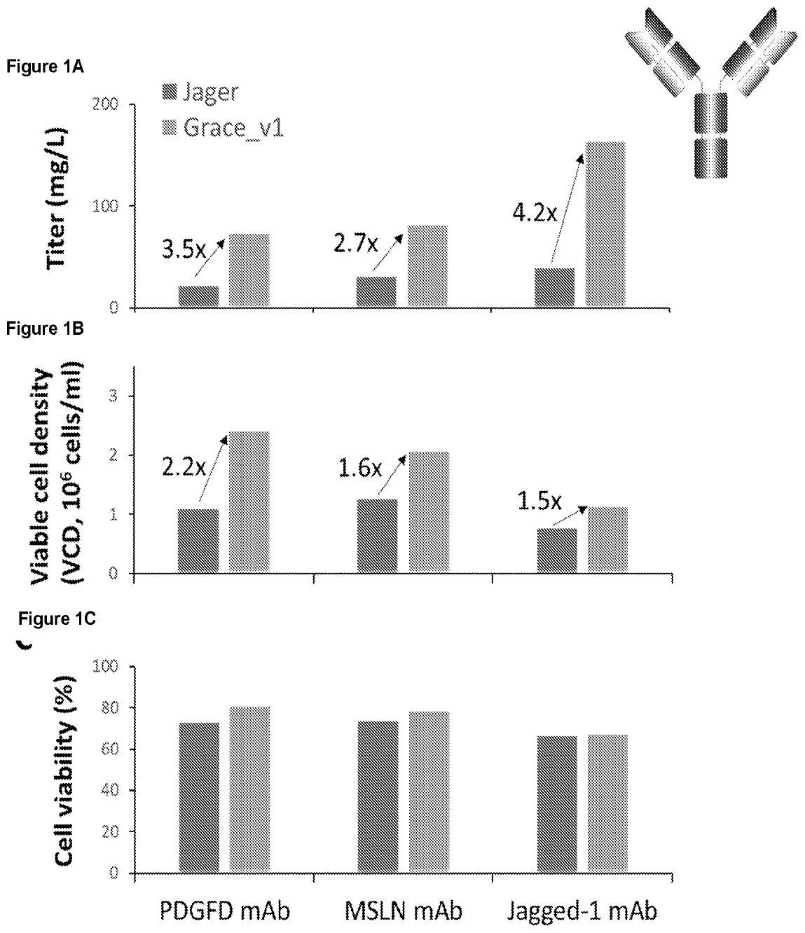

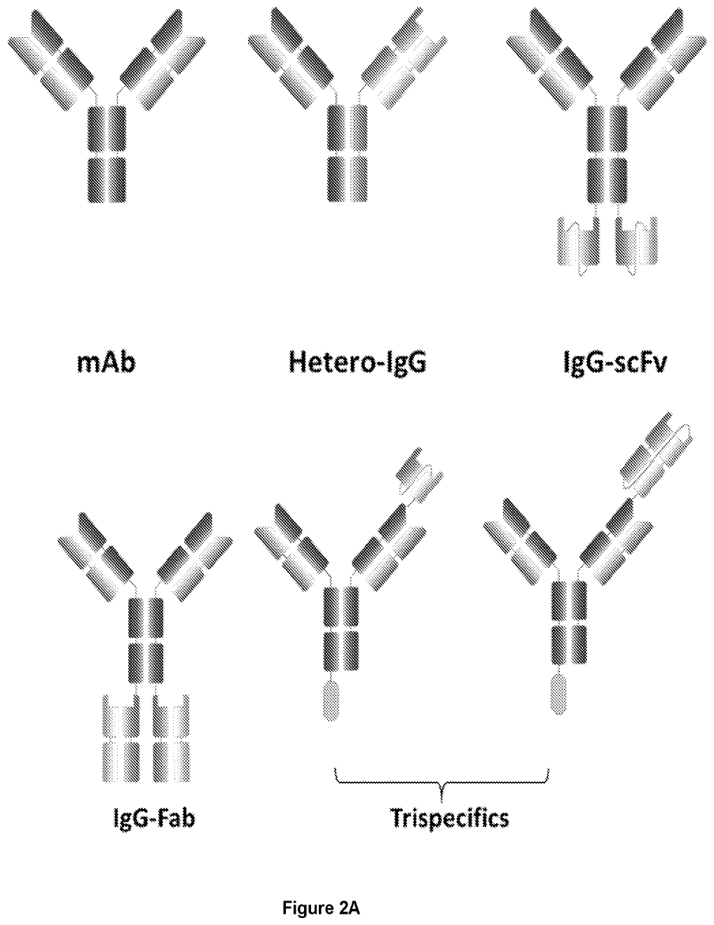

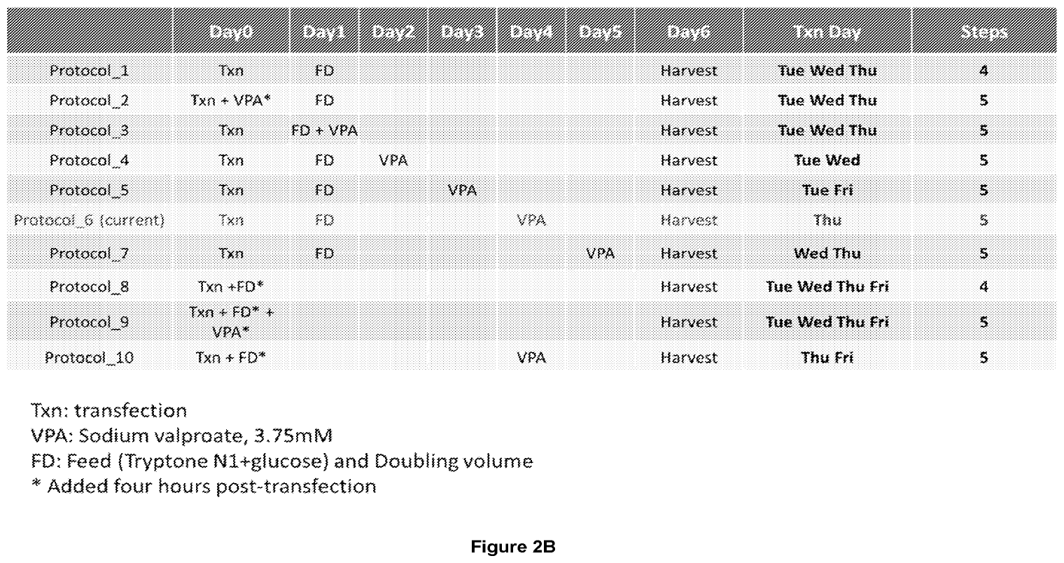

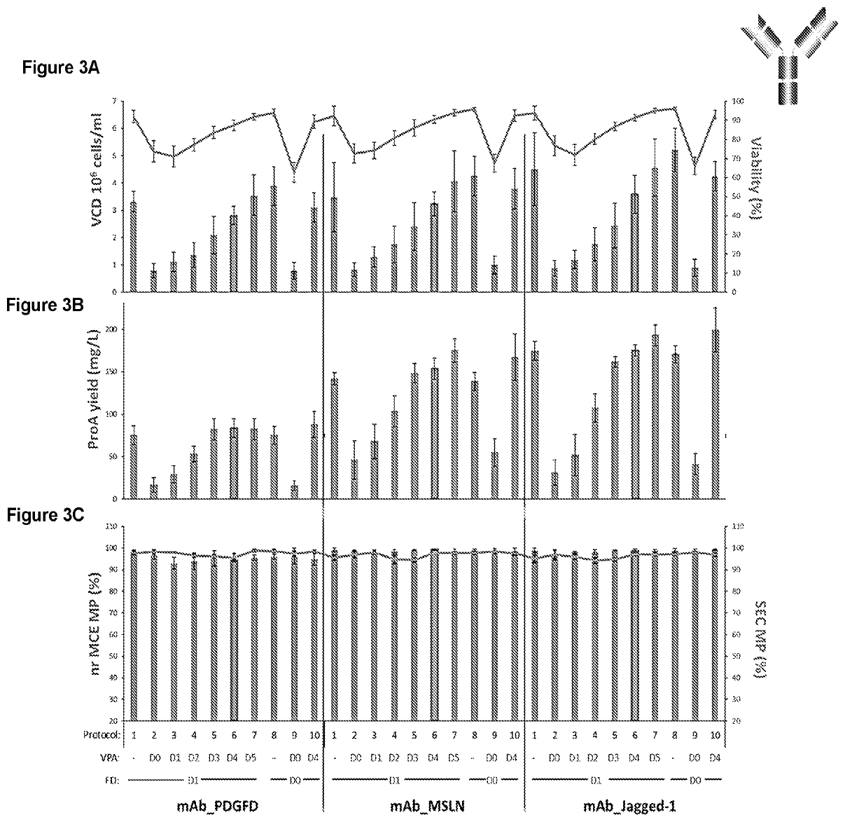

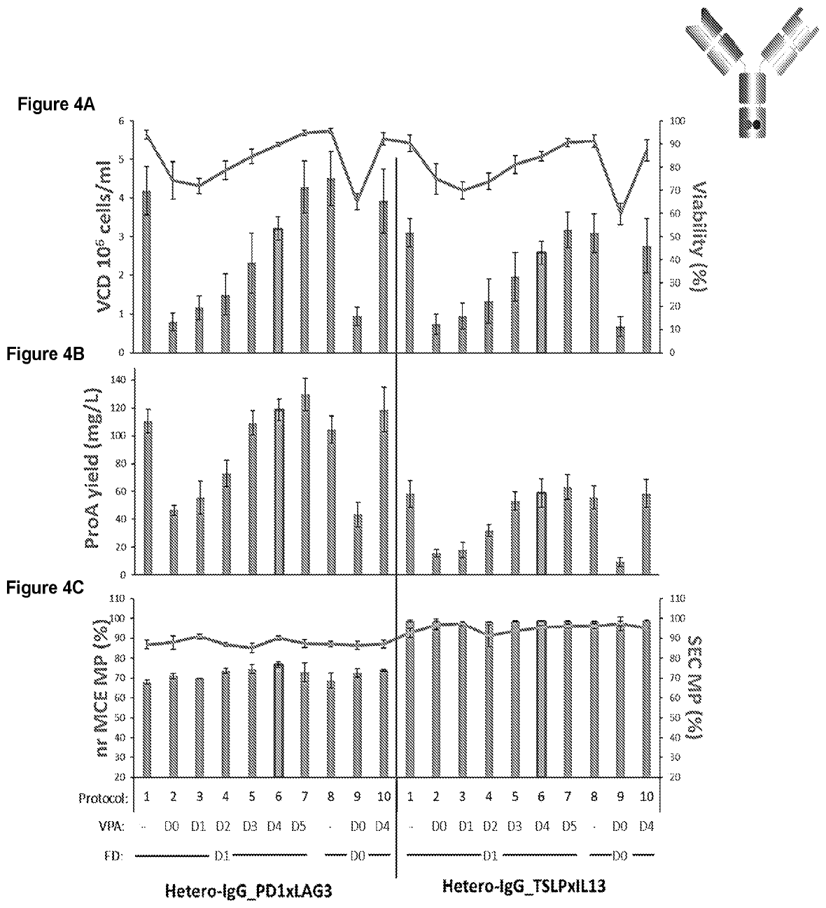

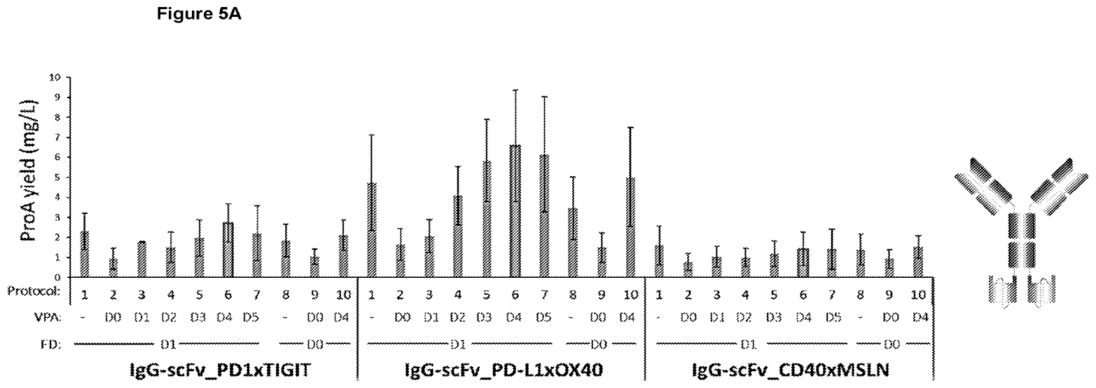

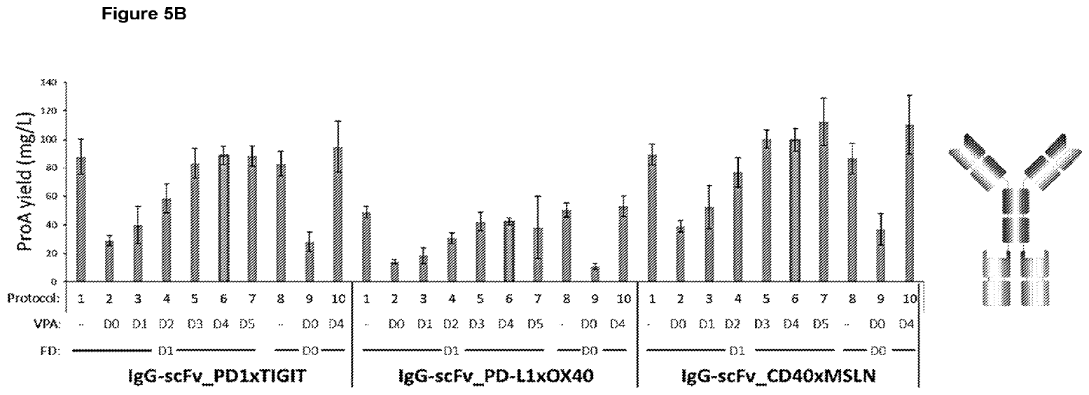

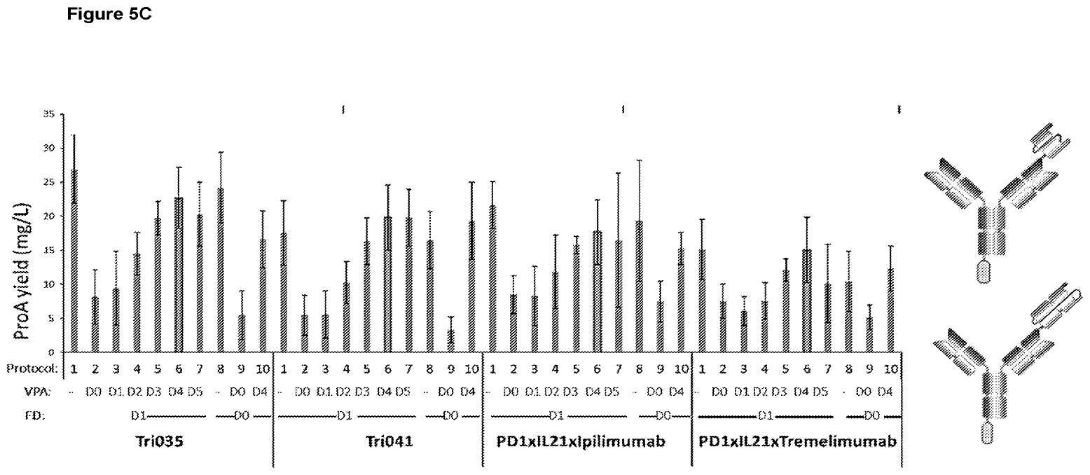

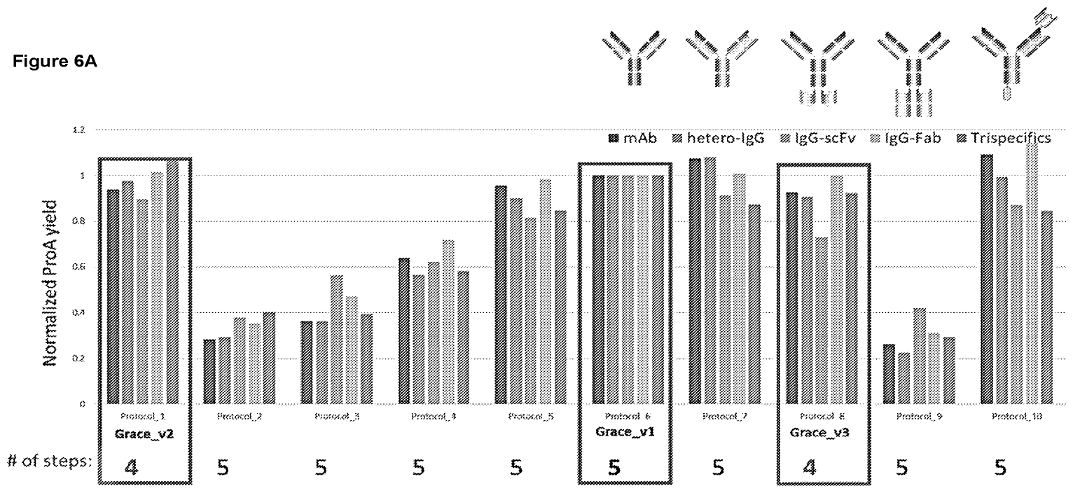

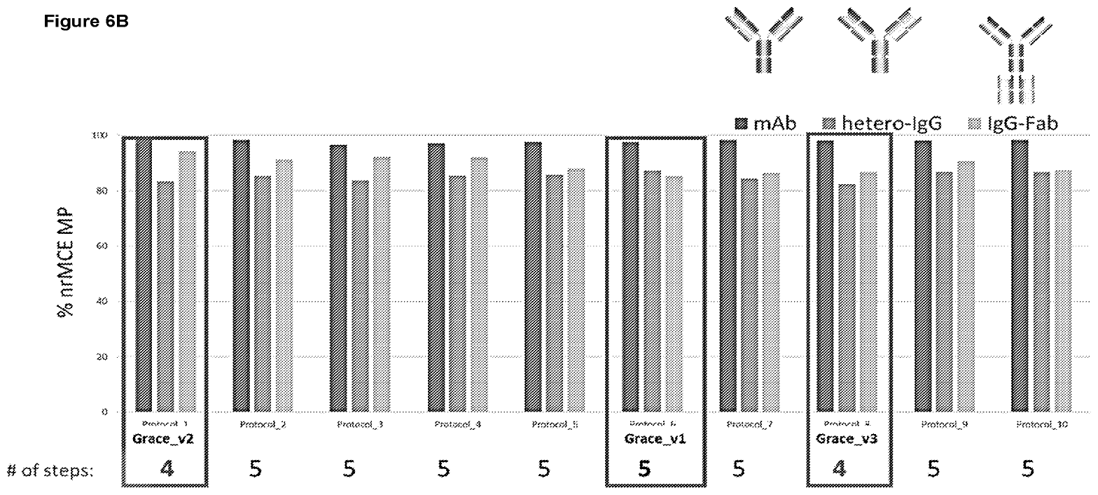

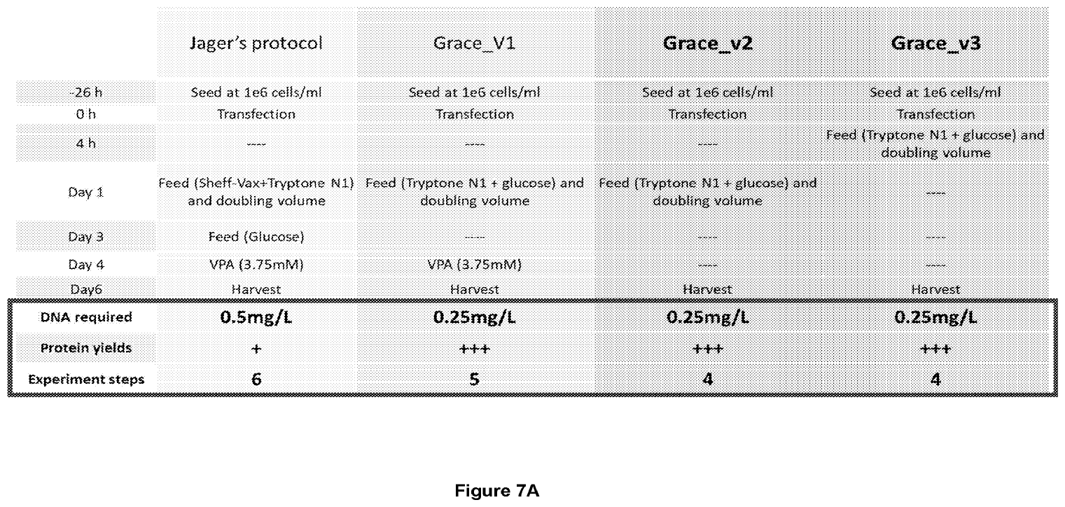

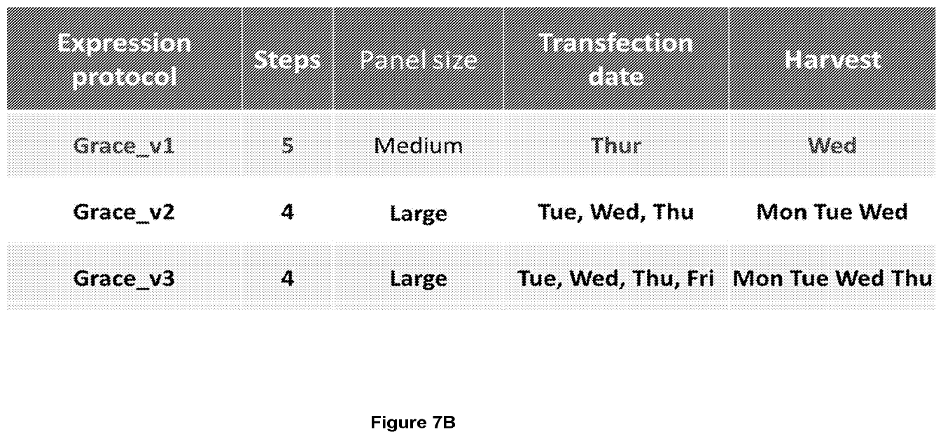

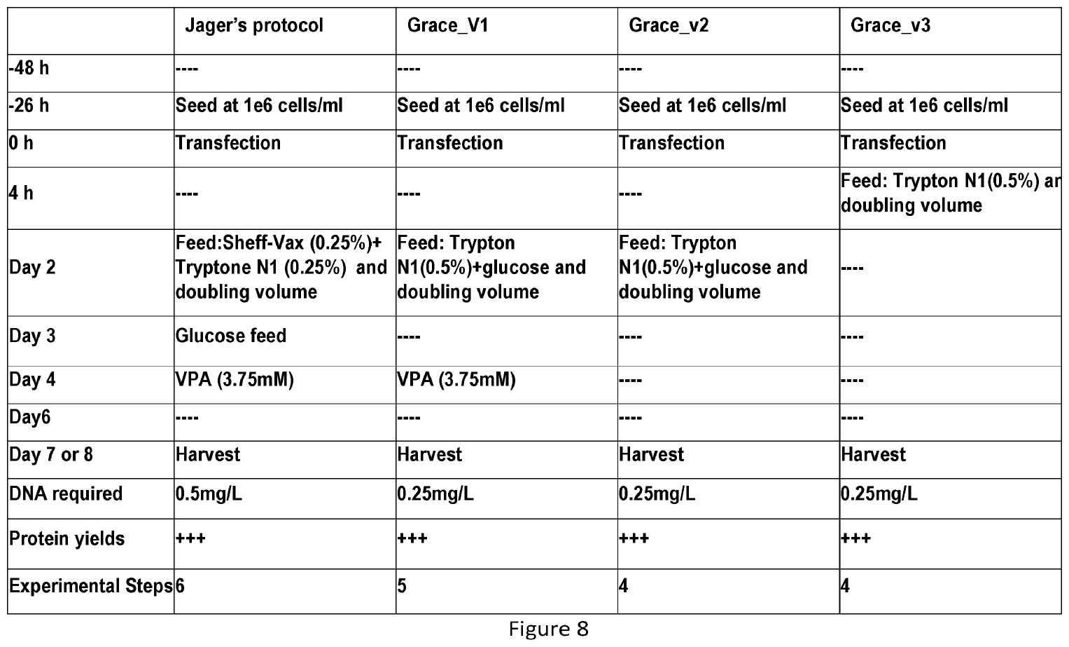

FIG. 3 depicts Comparison of 10 HEK293-6E protocols on expression of three mAbs. Grace_v1 (also named protocol 6) is highlighted in red color.
(A) Viable cell density (VCD) and percentage (%) at the time of harvest.
(B) Protein yield after HT kingfisher purification with ProA beads.
(C) Analysis of ProA purified proteins with non-reducing MCE and SEC. Intact IgGs appear as MP in MCE and SEC.

FIG. 4 depicts Comparison of 10 HEK293-6E protocols on expression of two Hetero-IgG bispecific antibodies. Grace_v1 (also named protocol_6) is highlighted in red color.
(A) Viable cell density (VCD) and percentage (%) at the time of harvest.
(B) Protein yield after HT kingfisher purification with ProA beads.
(C) Analysis of ProA purified proteins with non-reducing MCE and SEC. Intact IgGs appear as MP in MCE and SEC.

FIG. 5 depicts Comparison of 10 HEK293-6E protocols on expression of 3 IgG-scFv, 3 IgG-Fab and 4 Trispecifics. Current 293-6E protocol (#6) is highlighted in red color.
(A) Protein yield of 3 IgG-scFvs after HT kingfisher purification with ProA beads.
(B) Protein yield of 3 IgG-Fabs.
(C) Protein yield of 4 Trispecifics.

FIG. 6 depicts Miniaturized Golden Gate reaction increased ModVec efficiency. A) Schematic of the 11 kb 14-piece (including vector backbone) ModVec assembly. B) Golden Gate overhangs used for this assembly. Underlined overhangs were frequently observed to mispair with non-complementary overhang. C) Efficiency of ModVec assembly at different Golden Gate reaction volumes. Identification of two improved HEK293-6E protocols (Grace_v2 and Grace_v3) based on simplicity of experiment, ProA yield and product quality.
(A) Normalized ProA yield show that Grace_v2 and _v3 express comparable amount of proteins to Grace_v1. In addition, these two new protocols require one experimental step less than current one.
(B) Analysis of ProA purified proteins with non-reducing MCE show that two new protocols produce mAbs and Bispecifics with comparable quality as current protocol.

FIG. 7 depicts A Summary of HEK293-6E protocols including Jager's, Grace_v1, _v2 and _v3.
(A) Detail of protocols. Compared to Jager's protocol, Grace_v1 to _v3 increase protein yield, lower DNA for transfection, and reduce experiment steps (higher throughput).
(B) Grace_v2 and _v3 show higher throughput and more flexibility than Grace_v1.

FIG. 8 depicts a table summarizing the various methods of the claimed invention as compared to the Jager protocol.

### DETAILED DESCRIPTION OF THE INVENTION

The present invention is directed to a method for transfecting a population of cells with DNA. In one embodiment, the DNA comprises genomic DNA. In one embodiment, the DNA is a vector or plasmid. The present invention utilizes vectors comprising one or more nucleic acids encoding one or more components of multispecific antigen binding proteins (e.g. variable regions, light chains, heavy chains, modified heavy chains, and Fd fragments). The term “vector” refers to any molecule or entity (e.g., nucleic acid, plasmid, bacteriophage or virus) used to transfer protein coding information into a host cell. Examples of vectors include, but are not limited to, plasmids, viral vectors, non-episomal mammalian vectors and expression vectors, for example, recombinant expression vectors. The term “expression vector” or “expression construct” as used herein refers to a recombinant DNA molecule containing a desired coding sequence and appropriate nucleic acid control sequences necessary for the expression of the operably linked coding sequence in a particular host cell. An expression vector can include, but is not limited to, sequences that affect or control transcription, translation, and, if introns are present, affect RNA splicing of a coding region operably linked thereto. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals. A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired. In certain embodiments, a signal peptide is selected from the group consisting of MDMRVPAQLLGLLLLWLRGARC (SEQ ID NO: 1), MAWALLLLTLLTQGTGSWA (SEQ ID NO: 2), MTCSPLLLTLLIHCTGSWA (SEQ ID NO: 3), MEAPAQLLFLLLLWLPDTTG (SEQ ID NO: 4), MEWTWRVLFLVAAATGAHS (SEQ ID NO: 5), METPAQLLFLLLLWLPDTTG (SEQ ID NO: 6), MKHLWFFLLLVAAPRWVLS (SEQ ID NO: 7), and MEWSWVFLFFLSVTTGVHS (SEQ ID NO: 8).

Typically, expression vectors used in the host cells to produce multispecific antigen proteins will contain sequences for plasmid maintenance and for cloning and expression of exogenous nucleotide sequences encoding the components of the multispecific antigen binding proteins. Such sequences, collectively referred to as “flanking sequences,” in certain embodiments will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Each of these sequences is discussed below.

Optionally, the vector may contain a “tag”-encoding sequence, i.e., an oligonucleotide molecule located at the 5′ or 3′ end of the polypeptide coding sequence; the oligonucleotide tag sequence encodes polyHis (such as hexaHis), FLAG, HA (hemaglutinin influenza virus), myc, or another “tag” molecule for which commercially available antibodies exist. This tag is typically fused to the polypeptide upon expression of the polypeptide, and can serve as a means for affinity purification or detection of the polypeptide from the host cell. Affinity purification can be accomplished, for example, by column chromatography using antibodies against the tag as an affinity matrix. Optionally, the tag can subsequently be removed from the purified polypeptide by various means such as using certain peptidases for cleavage.

Flanking sequences may be homologous (i.e., from the same species and/or strain as the host cell), heterologous (i.e., from a species other than the host cell species or strain), hybrid (i.e., a combination of flanking sequences from more than one source), synthetic or native. As such, the source of a flanking sequence may be any prokaryotic or eukaryotic organism, any vertebrate or invertebrate organism, or any plant, provided that the flanking sequence is functional in, and can be activated by, the host cell machinery.

Flanking sequences useful in the vectors of this invention may be obtained by any of several methods well known in the art. Typically, flanking sequences useful herein will have been previously identified by mapping and/or by restriction endonuclease digestion and can thus be isolated from the proper tissue source using the appropriate restriction endonucleases. In some cases, the full nucleotide sequence of a flanking sequence may be known. Here, the flanking sequence may be synthesized using routine methods for nucleic acid synthesis or cloning.

Whether all or only a portion of the flanking sequence is known, it may be obtained using polymerase chain reaction (PCR) and/or by screening a genomic library with a suitable probe such as an oligonucleotide and/or flanking sequence fragment from the same or another species. Where the flanking sequence is not known, a fragment of DNA containing a flanking sequence may be isolated from a larger piece of DNA that may contain, for example, a coding sequence or even another gene or genes. Isolation may be accomplished by restriction endonuclease digestion to produce the proper DNA fragment followed by isolation using agarose gel purification, Qiagen7 column chromatography (Chatsworth, CA), or other methods known to the skilled artisan. The selection of suitable enzymes to accomplish this purpose will be readily apparent to one of ordinary skill in the art.

An origin of replication is typically a part of those prokaryotic expression vectors purchased commercially, and the origin aids in the amplification of the vector in a host cell. If the vector of choice does not contain an origin of replication site, one may be chemically synthesized based on a known sequence, and ligated into the vector. For example, the origin of replication from the plasmid pBR322 (New England Biolabs, Beverly, MA) is suitable for most gram-negative bacteria, and various viral origins (e.g., SV40, polyoma, adenovirus, vesicular stomatitus virus (VSV), or papillomaviruses such as HPV or BPV) are useful for cloning vectors in mammalian cells. Generally, the origin of replication component is not needed for mammalian expression vectors (for example, the SV40 origin is often used only because it also contains the virus early promoter).

A transcription termination sequence is typically located 3′ to the end of a polypeptide coding region and serves to terminate transcription. Usually, a transcription termination sequence in prokaryotic cells is a G-C rich fragment followed by a poly-T sequence. While the sequence is easily cloned from a library or even purchased commercially as part of a vector, it can also be readily synthesized using known methods for nucleic acid synthesis.

A selectable marker gene encodes a protein necessary for the survival and growth of a host cell grown in a selective culture medium. Typical selection marker genes encode proteins that (a) confer resistance to antibiotics or other toxins, e.g., ampicillin, tetracycline, or kanamycin for prokaryotic host cells; (b) complement auxotrophic deficiencies of the cell; or (c) supply critical nutrients not available from complex or defined media. Specific selectable markers are the kanamycin resistance gene, the ampicillin resistance gene, and the tetracycline resistance gene. Advantageously, a neomycin resistance gene may also be used for selection in both prokaryotic and eukaryotic host cells.

Other selectable genes may be used to amplify the gene that will be expressed. Amplification is the process wherein genes that are required for production of a protein critical for growth or cell survival are reiterated in tandem within the chromosomes of successive generations of recombinant cells. Examples of suitable selectable markers for mammalian cells include dihydrofolate reductase (DHFR) and promoterless thymidine kinase genes. Mammalian cell transformants are placed under selection pressure wherein only the transformants are uniquely adapted to survive by virtue of the selectable gene present in the vector. Selection pressure is imposed by culturing the transformed cells under conditions in which the concentration of selection agent in the medium is successively increased, thereby leading to the amplification of both the selectable gene and the DNA that encodes another gene, such as one or more components of the multispecific antigen binding proteins described herein. As a result, increased quantities of a polypeptide are synthesized from the amplified DNA.

A ribosome-binding site is usually necessary for translation initiation of mRNA and is characterized by a Shine-Dalgarno sequence (prokaryotes) or a Kozak sequence (eukaryotes). The element is typically located 3′ to the promoter and 5′ to the coding sequence of the polypeptide to be expressed. In certain embodiments, one or more coding regions may be operably linked to an internal ribosome binding site (IRES), allowing translation of two open reading frames from a single RNA transcript.

In some cases, such as where glycosylation is desired in a eukaryotic host cell expression system, one may manipulate the various pre- or prosequences to improve glycosylation or yield. For example, one may alter the peptidase cleavage site of a particular signal peptide, or add prosequences, which also may affect glycosylation. The final protein product may have, in the −1 position (relative to the first amino acid of the mature protein) one or more additional amino acids incident to expression, which may not have been totally removed. For example, the final protein product may have one or two amino acid residues found in the peptidase cleavage site, attached to the amino-terminus. Alternatively, use of some enzyme cleavage sites may result in a slightly truncated form of the desired polypeptide, if the enzyme cuts at such area within the mature polypeptide.

Expression and cloning vectors will typically contain a promoter that is recognized by the host organism and operably linked to the molecule encoding the polypeptide. The term “operably linked” as used herein refers to the linkage of two or more nucleic acid sequences in such a manner that a nucleic acid molecule capable of directing the transcription of a given gene and/or the synthesis of a desired protein molecule is produced. For example, a control sequence in a vector that is “operably linked” to a protein coding sequence is ligated thereto so that expression of the protein coding sequence is achieved under conditions compatible with the transcriptional activity of the control sequences. More specifically, a promoter and/or enhancer sequence, including any combination of cis-acting transcriptional control elements is operably linked to a coding sequence if it stimulates or modulates the transcription of the coding sequence in an appropriate host cell or other expression system.

Promoters are untranscribed sequences located upstream (i.e., 5′) to the start codon of a structural gene (generally within about 100 to 1000 bp) that control transcription of the structural gene. Promoters are conventionally grouped into one of two classes: inducible promoters and constitutive promoters. Inducible promoters initiate increased levels of transcription from DNA under their control in response to some change in culture conditions, such as the presence or absence of a nutrient or a change in temperature. Constitutive promoters, on the other hand, uniformly transcribe a gene to which they are operably linked, that is, with little or no control over gene expression. A large number of promoters, recognized by a variety of potential host cells, are well known. A suitable promoter is operably linked to the DNA encoding e.g., heavy chain, light chain, modified heavy chain, or other component of multispecific antigen binding proteins, by removing the promoter from the source DNA by restriction enzyme digestion and inserting the desired promoter sequence into the vector.

Suitable promoters for use with yeast hosts are also well known in the art. Yeast enhancers are advantageously used with yeast promoters. Suitable promoters for use with mammalian host cells are well known and include, but are not limited to, those obtained from the genomes of viruses such as polyoma virus, fowlpox virus, adenovirus (such as Adenovirus 2), bovine papilloma virus, avian sarcoma virus, cytomegalovirus, retroviruses, hepatitis-B virus and most preferably Simian Virus 40 (SV40). Other suitable mammalian promoters include heterologous mammalian promoters, for example, heat-shock promoters and the actin promoter.

Additional promoters which may be of interest include, but are not limited to: SV40 early promoter (Benoist and Chambon, 1981, Nature 290:304-310); CMV promoter (Thomsen et al., 1984, Proc. Natl. Acad. U.S.A. 81:659-663); the promoter contained in the 3′ long terminal repeat of Rous sarcoma virus (Yamamoto et al., 1980, Cell 22:787-797); herpes thymidine kinase promoter (Wagner et al., 1981, Proc. Natl. Acad. Sci. U.S.A. 78: 1444-1445); promoter and regulatory sequences from the metallothionine gene Prinster et al., 1982, Nature 296:39-42); and prokaryotic promoters such as the beta-lactamase promoter (Villa-Kamaroff et al., 1978, Proc. Natl. Acad. Sci. U.S.A. 75:3727-3731); or the tac promoter (DeBoer et al., 1983, Proc. Natl. Acad. Sci. U.S.A. 80:21-25). Also of interest are the following animal transcriptional control regions, which exhibit tissue specificity and have been utilized in transgenic animals: the elastase I gene control region that is active in pancreatic acinar cells (Swift et al., 1984, Cell 38:639-646; Omitz et al., 1986, Cold Spring Harbor Symp. Quant. Biol. 50:399-409; MacDonald, 1987, Hepatology 7:425-515); the insulin gene control region that is active in pancreatic beta cells (Hanahan, 1985, Nature 315: 115-122); the immunoglobulin gene control region that is active in lymphoid cells (Grosschedl et al., 1984, Cell 38:647-658; Adames et al., 1985, Nature 318:533-538; Alexander et al., 1987, Mol. Cell. Biol. 7: 1436-1444); the mouse mammary tumor virus control region that is active in testicular, breast, lymphoid and mast cells (Leder et al., 1986, Cell 45:485-495); the albumin gene control region that is active in liver (Pinkert et al., 1987, Genes and Devel. 1:268-276); the alpha-feto-protein gene control region that is active in liver (Krumlauf et al., 1985, Mol. Cell. Biol. 5: 1639-1648; Hammer et al., 1987, Science 253:53-58); the alpha 1-antitrypsin gene control region that is active in liver (Kelsey et al., 1987, Genes and Devel. 1: 161-171); the beta-globin gene control region that is active in myeloid cells (Mogram et al, 1985, Nature 315:338-340; Kollias et al, 1986, Cell 46:89-94); the myelin basic protein gene control region that is active in oligodendrocyte cells in the brain (Readhead et al., 1987, Cell 48:703-712); the myosin light chain-2 gene control region that is active in skeletal muscle (Sani, 1985, Nature 314:283-286); and the gonadotropic releasing hormone gene control region that is active in the hypothalamus (Mason et al., 1986, Science 234: 1372-1378).

An enhancer sequence may be inserted into the vector to increase transcription of DNA encoding a component of the multispecific antigen binding proteins (e.g., light chain, heavy chain, modified heavy chain, Fd fragment) by higher eukaryotes. Enhancers are cis-acting elements of DNA, usually about 10-300 bp in length, that act on the promoter to increase transcription. Enhancers are relatively orientation and position independent, having been found at positions both 5′ and 3′ to the transcription unit. Several enhancer sequences available from mammalian genes are known (e.g., globin, elastase, albumin, alpha-feto-protein and insulin). Typically, however, an enhancer from a virus is used. The SV40 enhancer, the cytomegalovirus early promoter enhancer, the polyoma enhancer, and adenovirus enhancers known in the art are exemplary enhancing elements for the activation of eukaryotic promoters. While an enhancer may be positioned in the vector either 5′ or 3′ to a coding sequence, it is typically located at a site 5′ from the promoter. A sequence encoding an appropriate native or heterologous signal sequence (leader sequence or signal peptide) can be incorporated into an expression vector, to promote extracellular secretion of the antibody. The choice of signal peptide or leader depends on the type of host cells in which the antibody is to be produced, and a heterologous signal sequence can replace the native signal sequence. Examples of signal peptides are described above. Other signal peptides that are functional in mammalian host cells include the signal sequence for interleukin-7 (IL-7) described in U.S. Pat. No. 4,965,195; the signal sequence for interleukin-2 receptor described in Cosman et al., 1984, Nature 312:768; the interleukin-4 receptor signal peptide described in EP Patent No. 0367 566; the type I interleukin-1 receptor signal peptide described in U.S. Pat. No. 4,968,607; the type II interleukin-1 receptor signal peptide described in EP Patent No. 0 460 846.

The expression vectors that are provided may be constructed from a starting vector such as a commercially available vector. Such vectors may or may not contain all of the desired flanking sequences. Where one or more of the flanking sequences described herein are not already present in the vector, they may be individually obtained and ligated into the vector. Methods used for obtaining each of the flanking sequences are well known to one skilled in the art. The expression vectors can be introduced into host cells to thereby produce proteins, including fusion proteins, encoded by nucleic acids as described herein.

In certain embodiments, nucleic acids encoding the different components of multispecific antigen binding proteins may be inserted into the same expression vector. In such embodiments, the two nucleic acids may be separated by an internal ribosome entry site (IRES) and under the control of a single promoter such that the light chain and heavy chain are expressed from the same mRNA transcript. Alternatively, the two nucleic acids may be under the control of two separate promoters such that the light chain and heavy chain are expressed from two separate mRNA transcripts.

Similarly, for IgG-scFv multispecific antigen binding proteins, the nucleic acid encoding the light chain may be cloned into the same expression vector as the nucleic acid encoding the modified heavy chain (fusion protein comprising the heavy chain and scFv) where the two nucleic acids are under the control of a single promoter and separated by an IRES or where the two nucleic acids are under the control of two separate promoters. For IgG-Fab multispecific antigen binding proteins, nucleic acids encoding each of the three components may be cloned into the same expression vector. In some embodiments, the nucleic acid encoding the light chain of the IgG-Fab molecule and the nucleic acid encoding the second polypeptide (which comprises the other half of the C-terminal Fab domain) are cloned into one expression vector, whereas the nucleic acid encoding the modified heavy chain (fusion protein comprising a heavy chain and half of a Fab domain) is cloned into a second expression vector. In certain embodiments, all components of the multispecific antigen binding proteins described herein are expressed from the same host cell population. For example, even if one or more components is cloned into a separate expression vector, the host cell is co-transfected with both expression vectors such that one cell produces all components of the multispecific antigen binding proteins.

After the vector has been constructed and the one or more nucleic acid molecules encoding the components of the multispecific antigen binding proteins described herein has been inserted into the proper site(s) of the vector or vectors, the completed vector(s) may be inserted into a suitable host cell for amplification and/or polypeptide expression. Thus, the present invention encompasses an isolated host cell comprising one or more expression vectors encoding the components of the multispecific antigen binding proteins. The term “host cell” as used herein refers to a cell that has been transformed, or is capable of being transformed, with a nucleic acid and thereby expresses a gene of interest. The term includes the progeny of the parent cell, whether or not the progeny is identical in morphology or in genetic make-up to the original parent cell, so long as the gene of interest is present. A host cell that comprises an isolated nucleic acid, preferably operably linked to at least one expression control sequence (e.g. promoter or enhancer), is a “recombinant host cell.”

A host cell, when cultured under appropriate conditions, synthesizes an antigen binding protein that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule.

Exemplary host cells include prokaryote, yeast, or higher eukaryote cells. Prokaryotic host cells include eubacteria, such as Gram-negative or Gram-positive organisms, for example, Enterobacteriaceae such as Escherichia, e.g., E. coli, Enterobacter, Erwinia, Klebsiella. Proteus, Salmonella, e.g., Salmonella typhimurium, Serratia, e.g., Serratia marcescans, and Shigella, as well as Bacillus, such as B. subtilis and B. licheniformis, Pseudomonas, and Streptomyces. Eukaryotic microbes such as filamentous fungi or yeast are suitable cloning or expression hosts for recombinant polypeptides. Saccharomyces cerevisiae, or common baker's yeast, is the most commonly used among lower eukaryotic host microorganisms. However, a number of other genera, species, and strains are commonly available and useful herein, such as Pichia, e.g. P. pastoris, Schizosaccharomyces pombe; Kluyveromyces, Yarrowia; Candida; Trichoderma reesia; Neurospora crassa; Schwanniomyces, such as Schwanniomyces occidentalis; and filamentous fungi, such as, e.g., Neurospora, Penicillium, Tolypocladium, and Aspergillus hosts such as A. nidulans and A. niger.

Host cells for the expression of glycosylated antigen binding proteins can be derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection of such cells are publicly available, e.g., the L-1 variant of Autographa californica NPV and the Bm-5 strain of Bombyx mori NPV.

Vertebrate host cells are also suitable hosts, and recombinant production of antigen binding proteins from such cells has become routine procedure. Mammalian cell lines available as hosts for expression are well known in the art and include, but are not limited to, immortalized cell lines available from the American Type Culture Collection (ATCC), including but not limited to Chinese hamster ovary (CHO) cells, including CHOK1 cells (ATCC CCL61), DXB-11, DG-44, and Chinese hamster ovary cells/-DHFR (CHO, Urlaub et al., Proc. Natl. Acad. Sci. USA 77: 4216, 1980); monkey kidney CV1 line transformed by SV40 (COS-7, ATCC CRL 1651); human embryonic kidney line (293 or 293 cells subcloned for growth in suspension culture (Graham et al., J. Gen Virol. 36: 59, 1977), including HEK293-6E cells; baby hamster kidney cells (BHK, ATCC CCL 10); mouse sertoli cells (TM4, Mather, Biol. Reprod. 23: 243-251, 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL51); TRI cells (Mather et al., Annals N.Y Acad. Sci. 383: 44-68, 1982); MRC 5 cells or FS4 cells; mammalian myeloma cells, and a number of other cell lines. In another embodiment, a cell line from the B cell lineage that does not make its own antibody but has a capacity to make and secrete a heterologous antibody can be selected. CHO cells are preferred host cells in some embodiments for expressing multispecific antigen binding proteins.

Host cells are transformed or transfected with the above-described nucleic acids or vectors for production of multispecific antigen binding proteins and are cultured in conventional nutrient media modified as appropriate for inducing promoters, selecting transformants, or amplifying the genes encoding the desired sequences. In addition, novel vectors and transfected cell lines with multiple copies of transcription units separated by a selective marker are particularly useful for the expression of antigen binding proteins. Thus, the present invention also provides a method for preparing a multispecific antigen binding protein described herein comprising culturing a host cell comprising one or more expression vectors described herein in a culture medium under conditions permitting expression of the multispecific antigen binding protein encoded by the one or more expression vectors; and recovering the multispecific antigen binding protein from the culture medium.

The host cells used to produce antigen binding proteins may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58: 44, 1979; Barnes et al., Anal. Biochem. 102: 255, 1980; U.S. Pat. Nos. 4,767,704; 4,657,866; 4,927,762; 4,560,655; or 5,122,469; WO90103430; WO 87/00195; or U.S. Pat. Re. No. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Upon culturing the host cells, the multispecific antigen binding protein can be produced intracellularly, in the periplasmic space, or directly secreted into the medium. If the antigen binding protein is produced intracellularly, as a first step, the particulate debris, either host cells or lysed fragments, is removed, for example, by centrifugation or ultrafiltration. The bispecific antigen binding protein can be purified using, for example, hydroxyapatite chromatography, cation or anion exchange chromatography, or preferably affinity chromatography, using the antigen(s) of interest or protein A or protein G as an affinity ligand. Protein A can be used to purify proteins that include polypeptides that are based on human γ1, γ2, or γ4 heavy chains (Lindmark et al., J. Immunol. Meth. 62: 1-13, 1983). Protein G is recommended for all mouse isotypes and for human γ3 (Guss et al., EMBO J. 5: 15671575, 1986). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the protein comprises a CH3 domain, the Bakerbond ABX™ resin (J. T. Baker, Phillipsburg, N.J.) is useful for purification. Other techniques for protein purification such as ethanol precipitation, Reverse Phase HPLC, chromatofocusing, SDS-PAGE, and ammonium sulfate precipitation are also possible depending on the particular multispecific antigen binding protein to be recovered.

As used herein, the term “antibody” refers to a tetrameric immunoglobulin protein comprising two light chain polypeptides (about 25 kDa each) and two heavy chain polypeptides (about 50-70 kDa each). The term “light chain” or “immunoglobulin light chain” refers to a polypeptide comprising, from amino terminus to carboxyl terminus, a single immunoglobulin light chain variable region (VL) and a single immunoglobulin light chain constant domain (CL). The immunoglobulin light chain constant domain (CL) can be kappa (κ) or lambda (λ). The term “heavy chain” or “immunoglobulin heavy chain” refers to a polypeptide comprising, from amino terminus to carboxyl terminus, a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4). Heavy chains are classified as mu (μ), delta (Δ), gamma (γ), alpha (α), and epsilon (ε), and define the antibody's isotype as IgM, IgD, IgG, IgA, and IgE, respectively. The IgG-class and IgA-class antibodies are further divided into subclasses, namely, IgG1, IgG2, IgG3, and IgG4, and IgA1 and IgA2, respectively. The heavy chains in IgG, IgA, and IgD antibodies have three domains (CH1, CH2, and CH3), whereas the heavy chains in IgM and IgE antibodies have four domains (CH1, CH2, CH3, and CH4). The immunoglobulin heavy chain constant domains can be from any immunoglobulin isotype, including subtypes. The antibody chains are linked together via inter-polypeptide disulfide bonds between the CL domain and the CH1 domain (i.e. between the light and heavy chain) and between the hinge regions of the antibody heavy chains.

In a human antibody, CH1 means a region having the amino acid sequence at positions 118 to 215 of the EU index. A highly flexible amino acid region called a “hinge region” exists between CH1 and CH2. CH2 represents a region having the amino acid sequence at positions 231 to 340 of the EU index, and CH3 represents a region having the amino acid sequence at positions 341 to 446 of the EU index.

“CL” represents a constant region of a light chain. In the case of a κ chain of a human antibody, CL represents a region having the amino acid sequence at positions 108 to 214 of the EU index. In a λ chain, CL represents a region having the amino acid sequence at positions 108 to 215.

Both the EU index as in Kabat et al., Sequences of Proteins of Immunological Interest, 5th Ed. Public Health Service, National Institutes of Health, Bethesda, MD (1991) and AHo numbering schemes (Honegger A. and Plückthun A. J Mol Biol. 2001 Jun. 8; 309(3):657-70) can be used in the present invention. Amino acid positions and complementarity determining regions (CDRs) and framework regions (FR) of a given antibody may be identified using either system. For example, EU heavy chain positions of 39, 44, 183, 356, 357, 360, 370, 392, 399, and 409 are equivalent to AHo heavy chain positions 46, 51, 230, 484, 485, 491, 501, 528, 535, and 551, respectively.

A “binding domain” or “BD”, may typically comprise an antibody light chain variable region (VL) and an antibody heavy chain variable region (VH); however, it does not have to comprise both. Fd fragments, for example, have two VH regions and often retain some antigen-binding function of the intact antigen-binding domain. Additional examples for the format of antibody fragments, antibody variants or binding domains include (1) a Fab fragment, a monovalent fragment having the VL, VH, CL and CH1 domains; (2) a F(ab′)2 fragment, a bivalent fragment having two Fab fragments linked by a disulfide bridge at the hinge region; (3) an Fd fragment having the two VH and CH1 domains; (4) an Fv fragment having the VL and VH domains of a single arm of an antibody, (5) a dAb fragment (Ward et al., (1989) Nature 341:544-546), which has a VH domain; (6) an isolated complementarity determining region (CDR), and (7) a single chain Fv (scFv), the latter being preferred (for example, derived from an scFV-library).

The disclosure of each reference set forth herein is incorporated herein by reference in its entirety.

The present invention is further illustrated by the following Examples:

### Examples

### Materials and Methods

### Cell Culture and Protein Expression

Suspension HEK293-6E cell line was licensed from National Research Council (NRC), Biotechnological Research Institute (BRI), Montreal, Canada. Cells are cultured in FreeStyle F-17 medium (Gibco, catalog #A1383502) supplemented with 0.1% Kolliphor P188 (Sigma, catalog #K4894), 25 g/ml G418 (Gibco, catalog #10131027) and 6 mM L-glutamine (Gibco, catalog #25030149). Cell density and viability were determined using a Beckman Coulter Vi-cell Cell Viability Analyzer based on the trypan blue exclusion method. During the maintenance and expansion stage, the suspension HEK293-6E cells were cultivated in 500 ml to 2.5 L polycarbonate flasks with 0.2 um ventilated caps (Corning) at 37° C., 5% CO2 atmosphere and gentle shaking at 120 rpm. When the cell density reached 2×10⁶ cells/ml, they were split to 3.5×10⁵ cells/ml for a 2 days passage and 2×10⁵ cells/ml for 3 days passage which required passage roughly three times per week.

To achieve a density of 2×10⁶ viable cells per ml for optimal transfection, cells with viability higher than 95% were seeded 26 hours in advance at a density of 1×10⁶ cells/ml. For transfection of each ml of cells, 0.5 μg DNA was complexed with 1.5 μl PEImax reagent (Polysciences, catalog #24765-2) in 100 μl FreeStyle F-17 medium for 10 min, and then added to cell culture. After transfection, cells were treated differently across different protocols. In Grace_v1, one day after transfection, cells were fed with Tryptone N1 solution (Organotechnie, catalog #19553) and glucose (Thermo Fisher, catalog #A2494001) to a final concentration of 5 g/L and 4.5 g/L, respectively, together with one additional volume of fresh cell culture medium. Three days later, 3.75 mM VPA (MP Biomedicals, catalog #0215206480) was further added with a hope to enhance protein expression. Protocols Grace_v2 and _v3 didn't require VPA, however, they still needed the addition of Trytone N1 and glucose at 4 hours or 24 hours post-transfection, respectively. For all protocols, condition medium was harvested for full purification with ProA and CEX columns at day 7 post transfection. For high-throughput kingfisher purification with ProA magnetic beads, 25 ul magnetic ProA beads (GE Life Sciences) was added to each ml of cell culture at day 6 post transfection, and then collected for purification at day 7.

### Plasmid Construction

The antibody HC and LC genes were synthesized by Twist Bioscience and then individually cloned into a mammalian transient expression vector using a Golden Gate assembly method.³ To reduce protein heterogenicity, all HCs were constructed with human IgG1 scaffold (IgG1-SEFL2) carrying an aglycosylation mutation and a novel engineered disulfide bond.⁴ For Hetero-IgGs that require HC heterodimerization, CPMs were introduced into the Fe regions. After sequencing confirmation by Sanger, transfection-grade DNA were prepared using Maxi plasmid purification kits (Qiagen, catalog #12165), and then mixed at a ratio of 1:1 (HC:LC) for monoclonal antibodies and IgG-scFv, 1:1:1:1 (HC1:LC1:HC2:LC2) for Hetero-IgGs, 1:1:1 (cLC:HC1:HC2) for IgG-Fab and Trispecifics.

High-Throughput Protein Purification with ProA Magnetic Beads

The KingFisher® Flex system (Thermo Fisher) was used for high-throughput protein purification with magnetic ProA beads (GE Life Sciences). Briefly, 4 ml 293-6E cells in 24-well deep blocks were transfected for protein expression, followed by addition of 100 μl magnetic ProA beads were added one day before harvest. Then, beads were collected and subjected to KingFisher purification with a 24 deep-well magnetic head. After washing 3 times with PBS and twice with Milli-Q water, proteins were eluted with 500 μl of 100 mM sodium acetate at pH 3.6 for 10 min, and then immediately neutralized by adding 10 μl of 3 M Tris, pH 11.0. Yield of purified proteins was measured by A280.

### Non-Reducing Micro Capillary Electrophoresis (MCE)

Purity of purified samples were analyzed with non-reducing MCE and analytical SEC. For non-reducing MCE, 6 μl of proteins were mixed with 21 μl of sample buffer (8.4 mM Tris-HCl pH 7.0, 7.98% Glycerol, 2.38 mM EDTA, 2.8% SDS and 2.4 mM Iodoacetamide), heated at 85° C. for 10 min, and then analyzed using a Caliper LabChip GXII Touch instrument (PerkinElmer).

We claim:

1. A method for transfecting a population of mammalian cells with DNA encoding a target molecule, the method comprising the following steps:
(a) providing the cells in cell culture media;
(b) performing a transfection step by contacting the cells with a liposome/DNA complex;
wherein the mg DNA:cell number ratio is about 0.25 mg:1×10⁹ cells;
(c) at about 40-56 hours after the transfection step, adding the following to the cell culture media in any order:
Tryptone N1 to a final concentration of about 4.5-5.5 g/L,
glucose to a final concentration of about 4.0-5.0 g/L, and
about 0.8-1.2 volumes of fresh cell culture media;
(d) at about 144-192 hours after the transfection step, harvesting the target molecule from the cell culture media.

2. The method according to claim 1, wherein the cells are suspension cells.

3. The method according to claim 1, wherein the cells are adherent cells.

4. The method according to claim 1, wherein the cells are selected from the group consisting of CHO cells, CHOK1 cells, DXB-11 cells, DG-44 cells, COS-7 cells, HEK293-6E cells, BHK cells, TM4 cells, CV1 cells, VERO-76 cells, HELA cells, MDCK cells, BRL 3A cells, W138 cells, Hep G2 cells, MMT cells, TRI cells, MRC 5 cells, and FS4 cells.

5. The method according to claim 1, wherein the cells are HEK293-6E cells.

6. The method according to claim 1, wherein the cells are seeded at about 1×10⁵-1×10⁷ cells/ml.

7. The method according to claim 6, wherein the cells are seeded at about 1×10⁶/ml.

8. The method according to claim 1, wherein the final concentration of Tryptone N1 is about 5.0 g/L.

9. The method according to claim 1, wherein the final concentration of glucose is about 4.5 g/L.

10. The method according to claim 1, wherein about 1 volume of fresh cell culture media is added.

11. The method according to claim 1, wherein at about 88-104 hours after the transfection step, valproic acid is added to a final concentration of about 3.5-4.0 mM.

12. The method according to claim 11, wherein the final concentration of valproic acid is about 3.75 mM.

13. The method according to claim 1, wherein step (c) is performed at about 48 hours after the transfection step.

14. The method according to claim 1, wherein step (d) is performed at about 168 hours after the transfection step.

15. The method according to claim 11, wherein the valproic acid is added at about 96 hours after the transfection step.

16. A method for transfecting a population of mammalian cells with DNA encoding a target molecule, the method comprising the following steps:
(a) providing the cells in cell culture media;
(b) performing a transfection step by contacting the cells with a liposome/DNA complex;
wherein the mg DNA:cell number ratio is about 0.25 mg:1×10⁹ cells;
(c) at about 2-6 hours after the transfection step, adding the following to the cell culture media in any order:
Tryptone N1 to a final concentration of about 4.5-5.5 g/L,
glucose to a final concentration of about 4.0-5.0 g/L, and
about 0.8-1.2 volumes of fresh cell culture media;
(d) at about 144-192 hours after the transfection step, harvesting the target molecule from the cell culture media.

17. The method according to claim 1, wherein the cells are suspension cells.

18. The method according to claim 1, wherein the cells are adherent cells.

19. The method according to claim 1, wherein the cells are selected from the group consisting of CHO cells, CHOK1 cells, DXB-11 cells, DG-44 cells, COS-7 cells, HEK293-6E cells, BHK cells, TM4 cells, CV1 cells, VERO-76 cells, HELA cells, MDCK cells, BRL 3A cells, W138 cells, Hep G2 cells, MMT cells, TRI cells, MRC 5 cells, and FS4 cells.

20. The method according to claim 1, wherein the cells are HEK293-6E cells.

21. The method according to claim 1, wherein the cells are seeded at about 1×10⁵-1×10⁷ cells/ml.

22. The method according to claim 6, wherein the cells are seeded at about 1×10⁶/ml.

23. The method according to claim 1, wherein the final concentration of Tryptone N1 is about 5.0 g/L.

24. The method according to claim 1, wherein the final concentration of glucose is about 4.5 g/L.

25. The method according to claim 1, wherein about 1 volume of fresh cell culture media is added.

26. The method according to claim 1, wherein at about 88-104 hours after the transfection step, valproic acid is added to a final concentration of about 3.5-4.0 mM.

27. The method according to claim 26, wherein the final concentration of valproic acid is about 3.75 mM.

28. The method according to claim 1, wherein step (c) is performed at about 4 hours after the transfection step.

29. The method according to claim 1, wherein step (d) is performed at about 168 hours after the transfection step.

30. The method according to claim 26, wherein the valproic acid is added at about 96 hours after the transfection step.

31. The method according to claim 1, wherein the target molecule is a multispecific antigen binding protein.
